(19) United States
(12) Patent Application Publication
(10) Pub. No.: US 2013/0165874 A1
(43) Pub. Date: Jun. 27, 2013

(54) TOILET PAPER USING POLYMERIC MATERIALS

(71) Applicants: Vladimir Vitalievich Miroshnichenko, Ljubertsi (RU); Vitaly Evgenievich Pilkin, Moscow (RU)

(72) Inventors: Vladimir Vitalievich Miroshnichenko, Ljubertsi (RU); Vitaly Evgenievich Pilkin, Moscow (RU)

(21) Appl. No.: 13/813,498

(22) PCT Filed: Jul. 21, 2011
(86) PCT No.: PCT/RU2011/000542

§ 371 Date: Jan. 31, 2013

(30) Foreign Application Priority Data
Aug. 27, 2010 (RU) 2010135702

(51) Int. Cl. A47K 10/16; A61M 35/00

(52) U.S. Cl. CPC A47K 10/16; A61M 35/00
USPC 604/289; 428/537.5; 428/211.1; 428/80; 428/84; 428/141; 428/43

(57) ABSTRACT

The invention can be used to produce environmentally friendly toilet paper in which the use of natural materials such as paper and/or cellulose and/or cotton is reduced. The essence of the first embodiment of the invention: the toilet paper using polymeric materials is a sheet made of at least one polymeric material and/or at least one material containing at least one polymeric material, herewith at least one side of specified sheet is coated completely or partially by material or material comprising paper and/or cellulose and/or cotton, herewith at least one polymeric material used in specified toilet paper is completely or partially water-soluble. The essence of the second embodiment of the invention: in the toilet paper using polymeric materials a part of a side of toilet paper sheet is coated with polymeric material and/or material containing polymeric material.

### CROSS-REFERENCE TO RELATED APPLICATIONS

This application is a U.S. national stage application of the PCT application PCT/RU2011/000542 filed on 21 Jul. 2011, published as WO 2012/026849, whose disclosure is incorporated herein in its entirety by reference, which PCT application claims priority of a Russian Federation application RU2010135702 filed on 27 Aug. 2010. The PCT application PCT/RU2011/000542 also contains parts of disclosure absent in the application RU2010135702.

### FIELD OF THE INVENTION

The invention relates to sanitary and hygienic facilities and can be used in the creation of environmentally friendly toilet paper with reduced use of natural materials such as paper and/or cellulose and/or cotton.

### BACKGROUND OF THE INVENTION

It is known toilet paper, which is used to maintain personal hygiene after defecation and/or after urination. More information is available at http://en.wikipedia.org/wiki/Toilet_paper. The disadvantage of this toilet paper is that toilet paper sheet is made up entirely of paper that leads to excessive consumption of paper, as only part of toilet paper is used directly for the intended purpose, and other part of toilet paper is used to not muck hands. As a result, a toilet paper user spends more toilet paper than it needs to maintain his sanitary and hygienic requirements.

It is known toilet paper for women (see patent FR2672788, abstract), in which one side of toilet paper is coated with water-resistant film made from materials such as colophony, gelatin, dextrin and latex, which are in the colloidal state, or made from water-resistant resins such as polyamide polyamine epichlorohydrin type. The above patent FR2672788 can be considered as the prototype of the first embodiment of the claimed invention. The disadvantage of this technical decision is that, being water-resistant, indicated film does not dissolve in water after using toilet paper, in which indicated film is used, and thus pollutes the environment.

It is known toilet paper (see U.S. Pat. No. 5,492,746, FIG. 1, column 1, lines 55-64), which consists of three layers of paper: two external and one internal, herewith outside of toilet paper to each of the external layer of paper in the center of toilet paper is attached by adhesive water-soluble, structurally strong paper coating with high absorption capacity, which does not permit hygienic problems. The disadvantage of this technical solution is not economical consumption of paper for manufacturing specified toilet paper. Technical solution proposed in the patent U.S. Pat. No. 5,492,746 can be considered as a prototype of the second embodiment of the claimed invention.

### DESCRIPTION OF THE INVENTION

The task to be solved and the technical result of the claimed technical decision in accordance with the first and the second embodiments of the invention is to create environmentally friendly toilet paper with reduced use of natural materials such as paper and/or cellulose and/or cotton.

With a view of correct understanding and interpretation of present invention the following terminology was used:

The central sector of toilet paper sheet is a toilet paper sheet sector, located on the side of a toilet paper sheet, which is coated by material or materials consisting of paper and/or cellulose and/or cotton or containing paper and/or cellulose and/or cotton, herewith specified sector has a round, or square, or rectangular, or triangular, or oval shape, or elongated oval shape, or rectangular shape with rounded edges, or any other geometric shape, inside of which (sector) the geometric center of toilet paper sheet is situated, in which outer sector of toilet paper sheet surrounds central sector of toilet paper sheet in whole or in part.

The outer sector of toilet paper sheet is a toilet paper sheet sector, which surrounds central sector of toilet paper sheet in whole or in part, herewith the area of outer sector of toilet paper sheet is equal to, or greater than, or less than the area of central sector of toilet paper sheet.

Specified technical result according to the first embodiment of the invention is achieved as follows:

Toilet paper using polymeric materials is a sheet made of at least one polymeric material and/or of at least one material containing at least one polymeric material, herewith at least one side of specified sheet is coated completely or partially by material or material comprising paper and/or cellulose and/or cotton (hereinafter material or material comprising paper and/or cellulose and/or cotton are referred to as “natural material”), herewith at least one polymeric material used in specified toilet paper is completely or partially water-soluble.

Side of toilet paper sheet coated with natural material may have two sectors: central sector and outer sector, herewith: a) the central sector is coated with natural material, while the outer sector is not coated with natural material, or b) the central sector and outer sector are coated with natural material, herewith according to “a” and “b” the outer sector of toilet paper sheet surrounds the central sector of toilet paper sheet in whole or in part.

The area of central sector of toilet paper sheet may be equal to, or greater than the area of outer sector of toilet paper sheet.

The area of central sector of toilet paper sheet may be less than the area of outer sector of toilet paper sheet.

Toilet paper may have a round, or square, or rectangular, or oval shape, or elongated oval shape, or rectangular shape with rounded edges.

Central sector of toilet paper sheet may have a round, or square, or rectangular, or triangular, or oval shape, or elongated oval shape, or rectangular shape with rounded edges.

Central sector and outer sector of toilet paper sheet may differ in density and/or surface texture and/or structure of natural material.

The area of toilet paper sheet can be more than 200 sq. cms. (square centimeters), or more than 150 sq. cms., or more than 100 sq. cms., or more than 50 sq. cms., or more than 30 sq. cms., or less than 30 sq. cms.

Natural material used in toilet paper sheet can be one-colored or multicolored.

Natural material used in the central sector and/or the outer sector of toilet paper sheet can be one-colored or multicolored.

Between sectors of toilet paper sheet may be indicated the border or part of the border, herewith specified designation can be carried out through at least one line of color different from colors of sectors of toilet paper sheet.

Natural material used in the central sector of toilet paper sheet may differ from natural material used in the outer sector of toilet paper sheet in tactile (touch) and/or in texture and/or in material or materials applied.

The polymeric material used in toilet paper sheet can be one-colored or multicolored.

At least one image and/or at least one decorative pattern and/or at least one character and/or at least one pattern and/or at least one geometric shape and/or at least one letter and/or at least one number and/or at least one hieroglyphic symbol can be placed: a) on side of toilet paper sheet which is coated by natural material, or b) on side of toilet paper sheet which is not coated by natural material, or c) on two sides of toilet paper sheet.

Toilet paper sheet can be impregnated with a fragrance and/or at least one medicinal agent and/or disinfectant.

Sheets of toilet paper can be interconnected to form a band, herewith a toilet paper sheet can be separated from other toilet paper sheet by perforation or punching.

A band, which consists of interconnected sheets of toilet paper, may be rolled, herewith the outer edge of band of toilet paper may be attached to the roll of toilet paper mechanically or by means of glue or thermal way, or may not be attached to the roll.

Inside the roll of toilet paper can be bung and/or hole.

Edge of band of toilet paper inside the roll of toilet paper can be attached to the bung mechanically or by means of glue or thermal way.

Coating a toilet paper sheet with natural material may be carried out in whole or in part by spraying, or mechanically or by means of glue or by compression or thermally or by using any combination of the above selected options.

Specified technical result according to the second embodiment of the invention is achieved as follows:

Toilet paper with polymeric materials is a sheet wherein a part of a side of said sheet is coated with polymeric material and/or material containing polymeric material (hereinafter polymeric material and/or material containing polymeric material referred to as “polymeric material”).

Polymeric material may be completely or partially water-soluble.

Toilet paper sheet can be made of material or materials containing paper and/or cellulose and/or cotton.

A part of a side of toilet paper sheet coated with polymeric material can be surrounded partially or in whole by another part of the same side of toilet paper sheet which is not coated by polymeric material.

A part of a side of toilet paper sheet coated with polymeric material can have a round, or square, or rectangular, or triangular, or oval shape, or elongated oval shape, or rectangular shape with rounded edges.

Toilet paper can have a round, or square, or rectangular, or oval shape, or elongated oval shape, or rectangular shape with rounded edges.

Coating a side of a toilet paper sheet with polymeric material may be carried out in whole or in part by spraying, or mechanically or by means of glue or by compression or thermally or by using any combination of the above selected options.

A part of a side of toilet paper sheet coated with polymeric material and another part of the same side of toilet paper sheet not coated with polymeric material may differ in tactile (touch) and/or in color.

Sheets of toilet paper can be interconnected to form a band, herewith a toilet paper sheet can be separated from other toilet paper sheet by perforation or punching.

Polymeric material can coat less than 95% of area of a side of toilet paper sheet, or less than 75% of area of a side of toilet paper sheet, or less than 50% of area of a side of toilet paper sheet, or less than 30% of area of a side of toilet paper sheet.

### EMBODIMENTS OF THE INVENTION

The first embodiment of the invention is technically feasible as in the prior art all the technical means, components and materials, that allow to produce toilet paper in accordance with the first embodiment of the invention, are known.

Significant features of the first embodiment of the invention, expressed in the section “Description of the invention” as an alternative for any choice permitted by this alternative in conjunction with other features characterizing the invention provide receiving the same technical result, which is to produce environmentally friendly toilet paper, in which use of natural materials such as paper and/or cellulose and/or cotton is reduced.

Possibility of receiving specified technical result for various combinations of characteristics of alternative features of the first embodiment of the invention is illustrated by the following non-limiting examples:

Example 1. Toilet paper is made of a polymeric material, one side of the toilet paper is partially coated with paper, herewith: a) side of toilet paper, partially coated with paper, has the central sector and the outer sector, b) the central sector is coated with paper, and the outer sector is not coated with paper, herewith the outer sector completely surrounds the central sector, c) both sectors have the same area, d) sheet of toilet paper is square, and the central sector of toilet paper has a round shape, e) the area of a sheet of toilet paper is more than 150 sq. cms., f) the material of the outer sector and the central sheet of toilet paper is one-colored, g) the material of the central sector of toilet paper sheet has a light brown color, h) the boundary between the central and the outer sector is designated by the line, which color differs from the color of sectors toilet paper sheet, i) polymeric material of toilet paper sheet is one-colored, j) on two sides of toilet paper sheet are placed the logo of the toilet paper manufacturer, k) the central sector of toilet paper sheet is impregnated with a fragrance, l) polymeric material used in toilet paper is completely water-soluble, m) sheets of toilet paper are interconnected to form a band, herewith a toilet paper sheet is separated from other toilet paper sheet by perforation, o) a band, which consists of interconnected sheets of toilet paper, is rolled, herewith inside the roll of toilet paper there is a bung, p) a sheet of toilet paper is attached to the bung by means of glue, q) the paper is applied to the central sector of toilet paper by spraying. After defecation a user of specified toilet paper puts his finger on that side of the toilet paper, which is made of a polymeric material. As a result, the opposite part of the toilet paper sheet, which is coated with paper is pressed against the anus, then moving the toilet paper sheet user wipes anus. The use of specified toilet paper can reduce the size of toilet paper, the density and the amount of paper used for toilet paper. In addition, toilet paper after falling into water, such as sewage, is completely dissolved, because the polymeric material used in the toilet paper is water-soluble.

Example 2. Toilet paper is made of a polymeric materials, one side of the toilet paper is completely coated with paper, herewith: a) toilet paper sheet has a rectangular shape, a) side of toilet paper, partially coated with paper, has the central sector and the outer sector, b) the area of a sheet of toilet paper is more than 200 sq. cms., c) side of toilet paper sheet coated with paper is multicolored, d) polymeric material used in toilet paper sheet is multicolored, e) on one side of toilet paper sheet advertising information is placed, f) on paper side of toilet paper sheet decorative pattern is placed, g) paper side of toilet paper sheet is impregnated with a disinfectant, h) polymeric material used in toilet paper is water-soluble, i) sheets of toilet paper are interconnected to form a band, herewith a toilet paper sheet is separated from other toilet paper sheet by punching, j) a band, which consists of interconnected sheets of toilet paper, is rolled, herewith inside the roll of toilet paper there is a bung, k) a sheet of toilet paper is attached to the bung by means of glue.

The second embodiment of the invention is technically feasible as in the prior art all the technical means, components and materials, that allow to produce toilet paper in accordance with the second embodiment of the invention, are known.

Significant features of the second embodiment of the invention, expressed in the section “Description of the invention” as an alternative for any choice permitted by this alternative in conjunction with other features characterizing the invention provide receiving the same technical result, which is to produce environmentally friendly toilet paper, in which use of natural materials such as paper and/or cellulose and/or cotton is reduced.

Possibility of receiving specified technical result for various combinations of characteristics of alternative features of the second embodiment of the invention is illustrated by the following non-limiting examples:

Example 1. Toilet paper sheet is made of natural material, for example, from paper. A part of a side of toilet paper sheet is coated with water-soluble material. A part of a side of toilet paper sheet coated with polymeric material has a round shape. A toilet paper sheet has a square shape. A part of a side of toilet paper sheet coated with polymeric material and another part of the same side of toilet paper sheet not coated with polymeric material differ each other in tactile (touch) and in color. Polymeric material coats 8% of area of a side of toilet paper sheet. After defecation a user of specified toilet paper puts his finger on that side of the toilet paper, which is made of a polymeric material. As a result, the opposite part of the toilet paper sheet, which is coated with paper is pressed against the anus, then moving the toilet paper sheet user wipes anus. The use of specified toilet paper can reduce the size of toilet paper, the density and the amount of paper used for toilet paper. In addition, toilet paper after falling into water, such as sewage, is completely dissolved, because the polymeric material used in the toilet paper is water-soluble.

### APPLICATIONS OF THE INVENTION

The first and the second embodiments of the claimed invention can be used in manufacturing environmentally friendly toilet paper, in which use of natural materials such as paper and/or cellulose and/or cotton is reduced.

1. A toilet paper using polymeric materials wherein a toilet paper is a sheet made of at least one polymeric material and/or at least one material containing at least one polymeric material, herewith at least one side of specified sheet is coated completely or partially by material or material comprising paper and/or cellulose and/or cotton (hereinafter material or material comprising paper and/or cellulose and/or cotton referred to as “natural material”), herewith at least one polymeric material used in specified toilet paper is completely or partially water-soluble.

2. The toilet paper according to claim 1, wherein side of toilet paper sheet coated with natural material has two sectors: central sector and outer sector, herewith: a) the central sector is coated with natural material, while the outer sector is not coated with natural material, or b) the central sector and outer sector are coated with natural material, herewith according to “a” and “b” the outer sector of toilet paper sheet surrounds the central sector of toilet paper sheet in whole or in part.

3. The toilet paper according to claim 1, wherein toilet paper has a round, or square, or rectangular, or oval shape, or elongated oval shape, or rectangular shape with rounded edges.

4. The toilet paper according to claim 2, wherein central sector of toilet paper sheet has a round, or square, or rectangular, or triangular, or oval shape, or elongated oval shape, or rectangular shape with rounded edges.

5. The toilet paper according to claim 2, wherein central sector and outer sector of toilet paper sheet differ in density and/or surface texture and/or structure of natural material.

6. The toilet paper according to claim 2, wherein between sectors of toilet paper sheet are indicated the border or part of the border.

7. The toilet paper according to claim 2, wherein natural material used in the central sector of toilet paper sheet differ from natural material used in the outer sector of toilet paper sheet in tactile (touch) and/or in texture and/or in materials applied.

8. The toilet paper according to claim 1, wherein toilet paper sheet is impregnated with a fragrance and/or at least one medicinal agent and/or disinfectant.

9. The toilet paper according to claim 1, wherein sheets of toilet paper interconnected to form a band are separated by perforation or punching.

10. The toilet paper according to claim 1, wherein coating a toilet paper sheet with natural material is carried out in whole or in part by spraying, or mechanically or by means of glue or by compression or thermally or by using any combination of the above selected options.

11. Toilet paper using polymeric materials wherein a part of a side of toilet paper sheet is coated with polymeric material and/or material containing polymeric material (hereinafter polymeric material and/or material containing polymeric material referred to as “polymeric material”).

12. The toilet paper according to claim 11, wherein polymeric material is completely or partially water-soluble.

13. The toilet paper according to claim 11, wherein toilet paper sheet is made of material or materials containing paper and/or cellulose and/or cotton.

14. The toilet paper according to claim 11, wherein a part of a side of toilet paper sheet coated with polymeric material is surrounded partially or in whole by another part of the same side of toilet paper sheet which is not coated by polymeric material.

15. The toilet paper according to claim 14, wherein a part of a side of toilet paper sheet coated with polymeric material has a round, or square, or rectangular, or triangular, or oval shape, or elongated oval shape, or rectangular shape with rounded edges.

16. The toilet paper according to claim 11, wherein toilet paper has a round, or square, or rectangular, or oval shape, or elongated oval shape, or rectangular shape with rounded edges.

17. The toilet paper according to claim 11, wherein coating a side of a toilet paper sheet with polymeric material is carried out in whole or in part by spraying, or mechanically or by means of glue or by compression or thermally or by using any combination of the above selected options.

18. The toilet paper according to claim 11, wherein a part of a side of toilet paper sheet coated with polymeric material and another part of the same side of toilet paper sheet not coated with polymeric material differ in tactile (touch) and/or in color.

19. The toilet paper according to claim 11, wherein sheets of toilet paper interconnected to form a band are separated by perforation or punching.

20. The toilet paper according to claim 11, wherein polymeric material coats less than 95% of area of a side of toilet paper sheet, or less than 75% of area of a side of toilet paper sheet, or less than 50% of area of a side of toilet paper sheet, or less than 30% of area of a side of toilet paper sheet.
